(19) United States
(12) Patent Application Publication
(10) Pub. No.: US 2026/0335040 A1
(43) Pub. Date: Oct. 29, 2026

(54) IMAGING DEVICE WITH TISSUE RETRACTION FEATURE

(71) Applicant: Arthrex, Inc., Naples, FL (US)

(72) Inventors: Ryan Kellar, Naples, FL (US); Matthew Johnson, Bonita Springs, FL (US); James Voos, Cleveland, OH (US); Rachel M. Frank, Denver, CO (US)

(73) Assignee: Arthrex, Inc., Naples, FL (US)

(21) Appl. No.: 19/656,111

(22) Filed: Apr. 23, 2026

Related U.S. Application Data
(60) Provisional application No. 63/795,863, filed on Apr. 28, 2025.

(51) Int. Cl. A61B 90/00; A61B 17/02

(52) U.S. Cl. CPC A61B 90/361; A61B 17/0218; A61B 2017/0225

(57) ABSTRACT

A surgical imaging apparatus includes an elongated band extending over a length from a first side to a second side with an intermediate section defining a compression surface formed therebetween. An imaging module is operably coupled to the intermediate section and configured to capture image data in a field-of-view directed from a viewing surface of the intermediate section.

### CROSS-REFERENCE TO RELATED APPLICATIONS

This application claims priority under 35 U.S.C. § 119(e) and the benefit of U.S. Provisional Application No. 63/795,863 entitled IMAGING DEVICE WITH TISSUE RETRACTION FEATURE, filed on Apr. 28, 2025, by Ryan Keller et al., the entire disclosure of which is incorporated herein by reference.

### BACKGROUND

Various surgical procedures may be implemented using minimally invasive techniques. Such techniques may require high levels of skill to successfully navigate internal cavities associated with the target anatomy without a direct view as in open procedures. Further, the manipulation of imaging devices and corresponding surgical tools for minimally invasive procedures may be cumbersome, and the corresponding manipulation may increase the risk of tissue damage. The disclosure provides for an apparatus and corresponding techniques that may improve visualization within the patient anatomy and assist in alleviating various challenges associated with minimally invasive procedures.

### SUMMARY

In various implementations, the disclosure may provide for an imaging apparatus that may be implemented in connection with an elongated band. As illustrated in various examples, the elongated band may be utilized to secure a camera module of the imaging apparatus to the anatomy of a patient. In addition to securing the imaging module to the patient, the elongated band of the imaging apparatus may also serve to retain and compress or retract tissue from a region of interest associated with a surgical procedure. The imaging module may be coupled to an intermediate section positioned between opposing sides of the elongated band. In this configuration, the imaging module may be configured to capture image data in a field-of-view directed from a viewing surface positioned along the elongated band and capture image data representative of the region of interest.

In some implementations, the elongated band may correspond to a compression band that may include mating features on the opposing sides that may be connected to enclose the elongated band about a compressible tissue of a patient. In various implementations, the first side of the elongated band may be operably coupled to a passing needle that may be introduced through an entry port to form a passage through the anatomy of the patient around the compressible tissue. A path formed by the passing needle may form a passage through which the elongated band may be drawn tensely through the entry port and outward through an exit port. In this configuration, the opposing sides of the elongated band may be connected to tighten the length of the band about the compressible tissue.

A compression surface may be formed along an intermediate section of the elongated band between the opposing sides. Approximately opposite the compression surface, a viewing surface may be formed along the intermediate section. The viewing surface may be associated with an origin of the field-of-view of the imaging module. In this configuration, the field-of-view of the imaging module may be directed approximately opposite the compression surface of the elongated band to capture image data in the field-of-view representative of the target anatomy associated with various surgical procedures.

These and other features, objects and advantages of the present disclosure will become apparent upon reading the following description thereof together with reference to the accompanying drawings.

### BRIEF DESCRIPTION OF THE DRAWINGS

FIG. 1A is a side cross-sectional view of a knee anatomy demonstrating a fat pad;

FIG. 1B is a projected view of a surgical imaging apparatus configured to be affixed to a portion of a patient anatomy;

FIG. 2 is a side cross-sectional view of a knee anatomy demonstrating a surgical imaging apparatus in connection with a compressible tissue;

FIG. 3 is a detailed side cross-sectional view of a surgical imaging apparatus;

FIG. 4 is a projected view of a multi-camera imaging module implementation of the surgical imaging apparatus;

FIG. 5 is a projected view of a multi-camera imaging module implementation of the surgical imaging apparatus comprising an interconnected portions;

FIG. 6A is a projected view demonstrating a step of a surgical procedure introducing a surgical imaging device in connection with an elongated band;

FIG. 6B is a projected view demonstrating a step of a surgical procedure introducing a surgical imaging device in connection with an elongated band;

FIG. 6C is a projected view demonstrating a step of a surgical procedure introducing a surgical imaging device in connection with an elongated band;

FIG. 6D is a projected view demonstrating a step of a surgical procedure introducing a surgical imaging device in connection with an elongated band;

FIG. 7 is a flow chart demonstrating a method for implementing a surgical imaging device in a minimally invasive procedure; and

FIG. 8 is a block diagram demonstrating an imaging system in accordance with the disclosure.

### DETAILED DESCRIPTION

In the following description, reference is made to the accompanying drawings, which show specific implementations that may be practiced. Wherever possible, the same reference numbers will be used throughout the drawings to refer to the same or like parts. It is to be understood that other implementations may be utilized, and structural and functional changes may be made without departing from the scope of this disclosure.

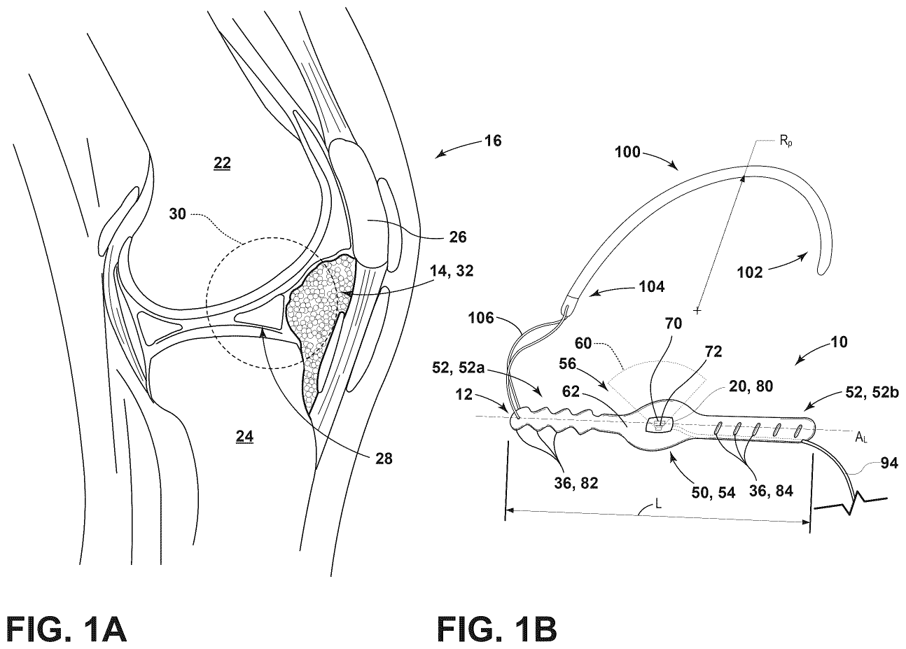

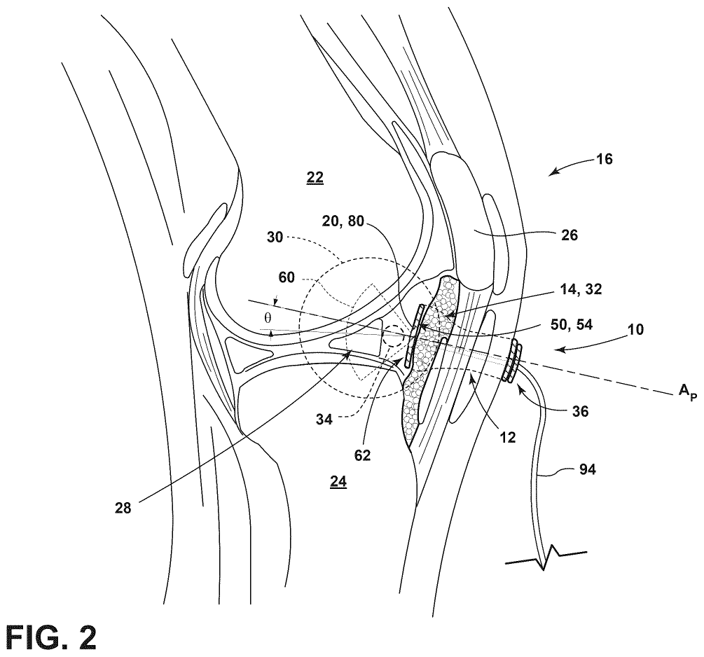

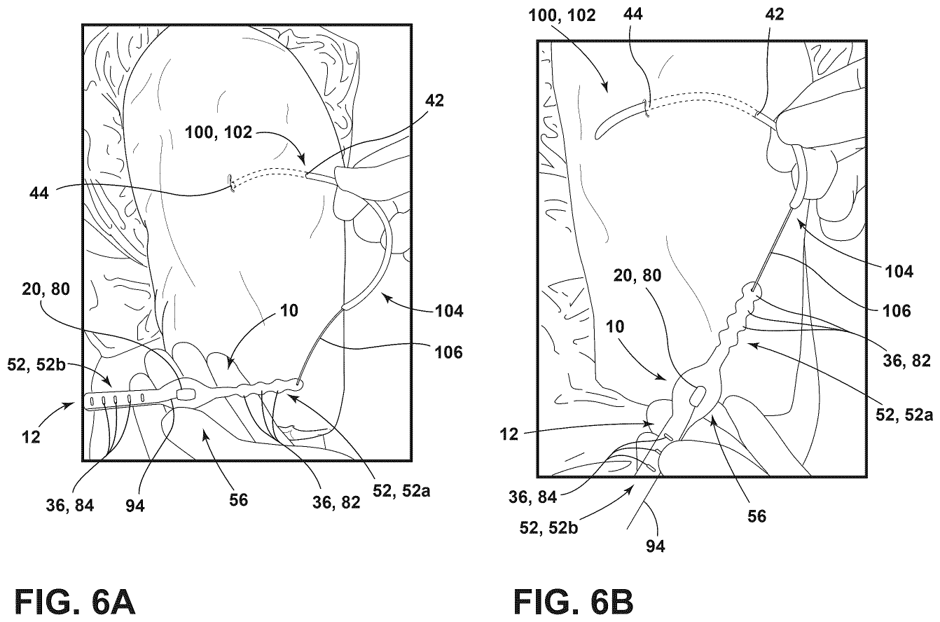

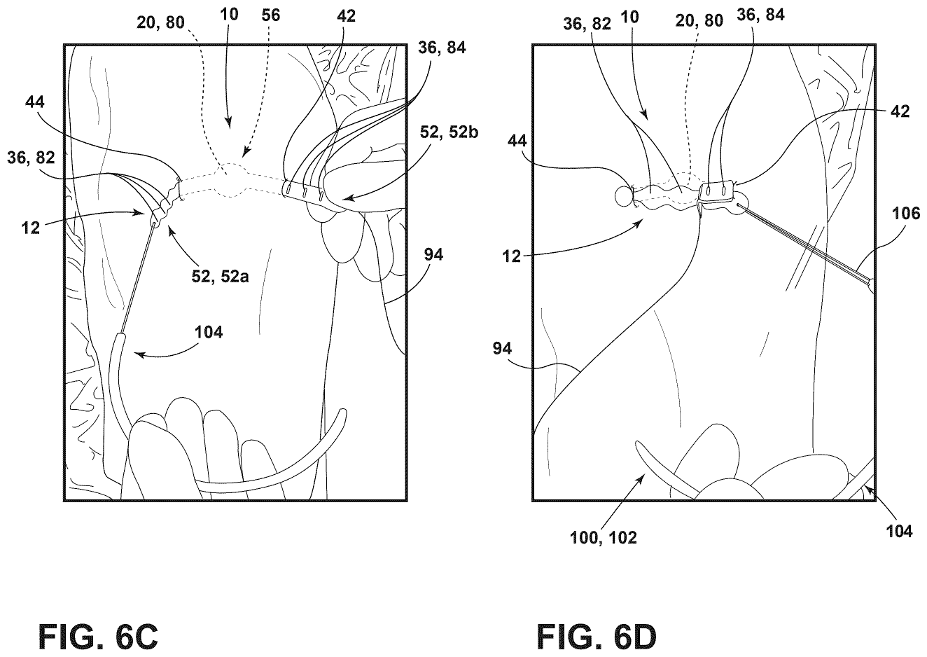

Referring generally to FIGS. 1 and 2, a surgical imaging apparatus 10 may comprise a passthrough attachment feature in the form of an elongated band 12. In the example shown, the surgical imaging apparatus 10 may be shown in various states of deployment in reference to a compressible tissue 14 of a knee joint 16. However, it shall be understood that the imaging apparatus 10 may be implemented in a variety of applications to support the engagement of an imaging module 20 fixated to a corresponding portion of the anatomy of a patient to support various surgical procedures, particularly minimally invasive procedures. As demonstrated in FIGS. 1A and 2, a cross-sectional view of the anatomy of the knee joint 16 may be shown demonstrating a femur 22, tibia 24, and patella 26, as well as the cartilage 28 separating the bony anatomy. In the knee joint 16 as well as various portions of the anatomy, compressible tissue 14 may occupy an interior space that may correspond to a region of interest 30 for a surgical procedure. In the specific example shown, the compressible tissue 14 may correspond to an infrapatellar fat pad or fat pad 32 positioned adjacent to the cartilage 28. In operation, the elongated band 12 may be deployed through a passage 34 and enclosed about the compressible tissue 14 via one or more retention fasteners 36 to withdraw the compressible tissue 14 from the region of interest 30, as shown in FIG. 2. In this configuration, various surgical tools may access the region of interest via an entry port 42 or an exit port 44 (see FIGS. 6A-6D) to conduct procedures on the anatomy associated with the region of interest 30.

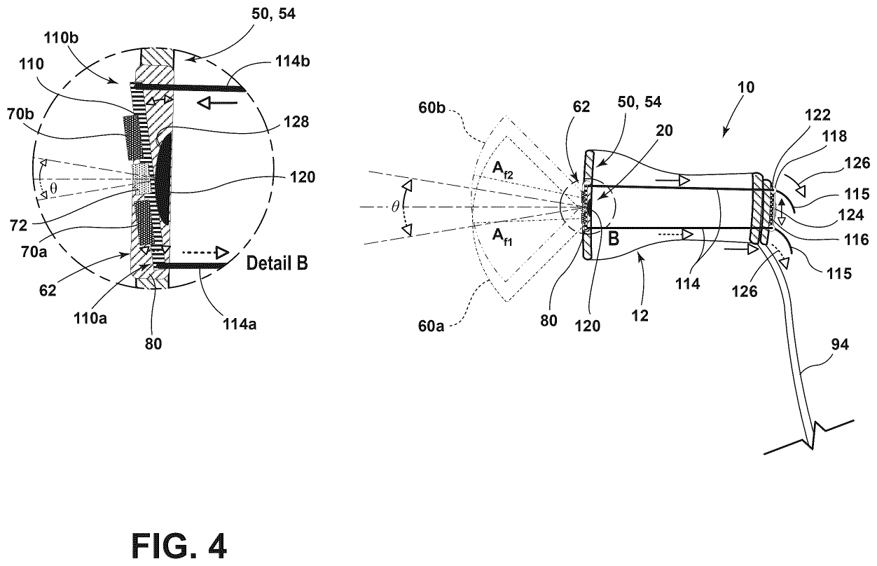

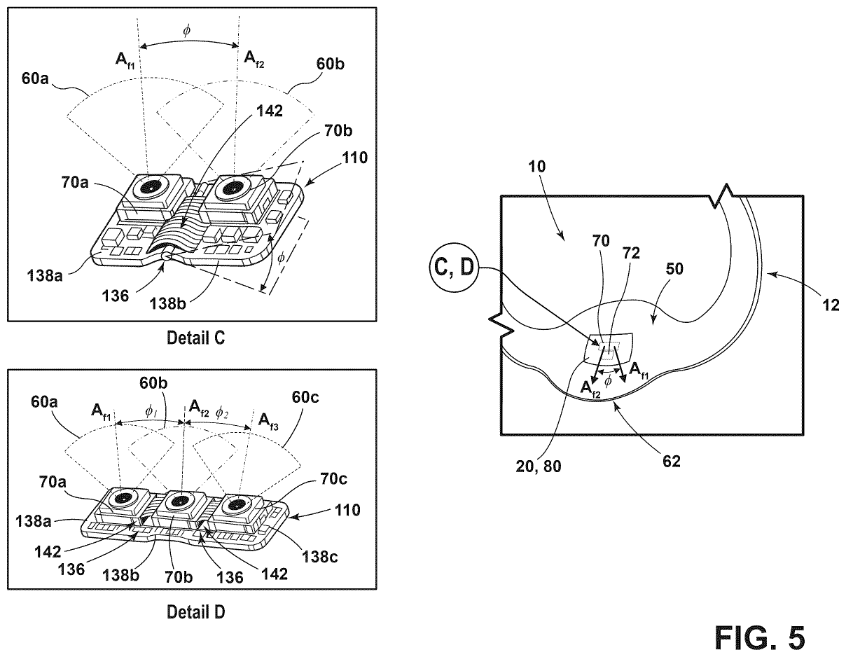

The specific steps associated with a method for deployment of the surgical imaging apparatus 10 to engage the compressible tissue 14 are described later in references to FIG. 4-5. Still referring to FIGS. 1-2, in various implementations, the elongated band 12 may form a compression surface 50 formed between opposing sides 52 of the elongated band 12. For example, the compression surface 50 may correspond to an expanded region of surface area 54 that may be positioned between a first side 52a and a second side 52b positioned on the opposing sides 52 of the elongated band 12. In this configuration, the imaging module 20 may be operably coupled to an intermediate section 56 of the elongated band 12 and configured to capture image data in a field-of-view 60 directed from a viewing surface 62 directed into the anatomy of the patient toward the region of interest 30. In various implementations, the viewing surface 62 and corresponding direction of the field-of-view 60 may be directed substantially opposite the compression surface 50. In this configuration, the positioning of the compression surface 50 to withdrawal the compressible tissue 14 from the region of interest 30 may align the field-of-view 60 to provide effective visualization of the region of interest 30 for various surgical procedures.

Though described as being directed approximately opposite the compression surface 50, the field-of-view 60 and/or the viewing surface 62 may be oriented at an imager angle θ relative to a projection axis A_{P} that may be defined perpendicular to the compression surface 50. In this way, an imager 70 and/or light source 72 of the imaging module 20 may be angled to align the field-of-view 60 in a variety of orientations relative to the compression surface 50. In this way, the imaging module 20 may provide for the engagement of the elongated band 12 to the compressible tissue 14.

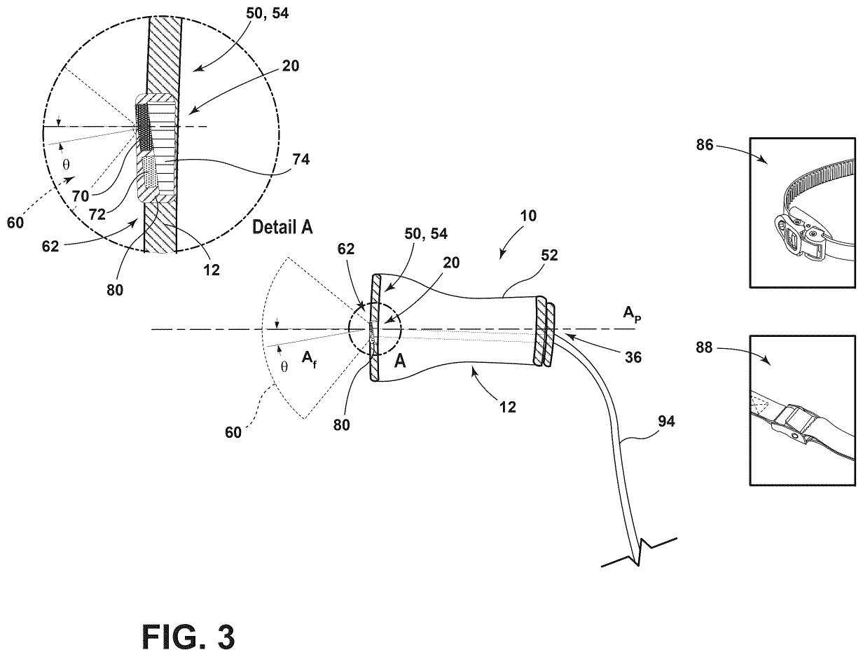

As best illustrated in FIGS. 2 and 3, the imager 70 and/or the light source 72 may be oriented at an imager angle θ that may range from approximately 0° to 90° or more. For example, the imager angle θ may correspond to various angles including, but not limited to, the following: 0°, 10°, 15°, 20°, 30°, 45°, 60°, 75°etc. Accordingly, in various implementations, emissions of light output from the light source 72 may illuminate the field-of-view 60 along the imager angle θ. The imager 70 and/or light source 72 may be oriented in an angled configuration relative to the compression surface 50 via an angled insert 74 that may form an interchangeable portion of the image module 20. In this configuration, the insert 74 and/or other portions of the imaging module 20 (e.g., the entire module) may correspond to interchangeable features of the imaging apparatus 10 that may be modified or replaced to adapt the apparatus 10 to a variety of procedures. Accordingly, the imaging apparatus 10 may be implemented in a variety of configurations to suit the needs of a patient or the preferences of the user.

Still referring to FIG. 3, the imager 70 may include one or more imagers (e.g., image sensors, optical sensors, depth sensors, motion sensors, etc.), which may be implemented to capture the image data and corresponding depth or surface profile information within the field-of-view 60. In some implementations, the sensors may include one or more cameras or imagers, which may correspond to a stereoscopic imager. For example, one or more cameras or optical assemblies may be incorporated in the imaging module 20 and may be separated over a distance on the viewing surface 62. The one or more imagers 70 may be configured to capture light in one or more ranges of wavelengths or spectrums including, but not limited to, a visible light spectrum ranging from approximately 380 nm to 700 nm and a near infrared (NIR) light spectrum ranging from approximately 760 nm to 2500 nm. Similarly, the light source 72 may include one or more light sources, which may be incorporated in the imaging module 20. The one or more light sources 72 may be configured to emit light in corresponding ranges of wavelengths, including the visible light spectrum and the NIR spectrum that may be readily detected by the one or more imagers 70 (e.g., the stereoscopic imager) of the imaging module 20. In this way, the camera apparatus 10 may be configured to capture image and depth data associated with the region of interest 30 to assist with visualization for a variety of procedures.

As shown in FIGS. 1B and 3, the imaging module 20 may be at least partially encapsulated and/or formed within a pocket 80 formed by the intermediate section 56 of the elongated band 12. The elongated band 12 may be formed from a variety of materials that may be suited to a clinical or surgical environment. For example, the elongated band may be formed from various polymeric materials including, but not limited to, silicones and/or thermoplastic elastomers and include various features, such as the retention fasteners 36 and/or pocket 80, to implement the surgical imaging apparatus 10 in a variety of configurations.

As demonstrated in FIG. 1B, the retention fasteners 36 may be implemented on the opposing sides 52 of the elongated band 12. For example, the retention fasteners may include at least one engaging rib 82 on the first side 52a, and at least one receiving slot 84 on the second side 52b. In operation, the at least one receiving slot 84 may be configured to receive the at least one engaging rib 82 to secure the length L of the band 12 about the compressible tissue 14. In the example shown, the at least one engaging rib 82 and the at least one receiving slot 84 may correspond to a plurality of engaging ribs 82 and receiving slots 84 distributed along the length L on the opposing sides 52. In this configuration, the portion of the length L of the band 12 enclosed about the compressible tissue 14 may be adjusted to suit the desired compression for various procedures and patients.

Referring still to FIGS. 1-3, in some implementations, the engaging ribs 82 may correspond to or form wings that protrude laterally outward from a longitudinal axis A_{L} along a length L of the elongated band 12. Though demonstrated as the slots 84 and corresponding ribs 82, the retention fasteners 36 may correspond to various features configured to maintain tension of the elongated band 12 along the length L enclosed about the compressible tissue 14. For example, the retention fasteners 36 may be implemented as a ladder strap 86, cam buckle 88, or various other fastener arrangements. In addition to the specific configurations shown, the features of the retention fasteners 36 may be combined in various ways to suit a variety of applications.

Referring again to FIG. 1B, the imager 70 and/or light source 72 of the imaging module 20 may be in conductive communication with a control console 90 of an imaging system 92 via a conductive connection 94. For example, the conductive connection 94 may extend through the second side 52b of the elongated band 12 opposite a passing needle 100 operably couple to the first side 52a. The conductive connection 94 may correspond to a cable interface that may be molded within or otherwise affixed to the elongated band 12. As later discussed in reference to FIGS. 6A-6D, the passing needle 100 may be interconnected to the first side 52a of the elongated band 12 and configured to define a path of the passage 34 through the anatomy exemplified as the knee joint 16 between the entry port 42 and the exit port 44. In various implementations, the passing needle 100 may correspond to a curved structure extending along a predefined radius Rₚ and may form a blunt tip or bullet-shaped tip at a distal end portion 102. Opposite the distal end portion 102, a proximal end portion 104 may be interconnected to the first side 52a of the elongated band 12 via a flexible coupling 106. In this configuration, the passing needle 100 may be utilized to define a path of the passage 34 proximal to the region of interest 30 and further draw the elongated band 12 of the surgical imaging apparatus 10 through the passage 34 to provide visualization within the field-of-view 60 to support various surgical procedures. Once passed through the tissue to define the passage 34, the flexible coupling 106 may be cut and the passing needle 100 may be discarded.

Referring generally to FIGS. 4 and 5, in some implementations, the imaging module 20 may comprise a multi-camera imaging module that includes a plurality of imagers or cameras 70 (e.g., cameras 70a, 70b) arranged on a mounting plate or structure 110 (e.g., PCB or base plate). As illustrated in FIGS. 4 and 5, the imagers 70 may be positioned in a linear array, a matrix array, a stereoscopic pair, or other configurations such that corresponding fields of view 60 (e.g., 60a, 60b) may overlap or diverge to provide varying coverage in the corresponding fields-of-view 60.

Referring first to FIG. 4, in various implementations, an orientation of the mounting structure 110 (and therefore the pitch of the imagers 70 and the corresponding fields of view 60) may be selectively adjusted by one or more tensioning members in the form of tension lines 114 (e.g., a first tension line 114a and a second tension line 114b). The tension lines 114 may be affixed to opposing end portions 110a, 110b of the mounting structure 110. For example, as shown, two opposing tension lines 114a, 114b may be connected to the opposing end portions 110a, 110b of the mounting structure 110. In this configuration, application of a tensioning force 126 along either of the opposing tension lines 114 may draw the corresponding connected end portion 110a or 110b and cause the mounting structure 110 to pivot at an adjustment angle θ. As a result, the connected imagers 70 and corresponding fields of view 60 may adjust in pitch in conjunction with the adjustment angle θ. In this way, a user may selectively apply tension to either of the tension lines 114 to cause the mounting structure 110 to pivot toward the applied tension.

As demonstrated in FIG. 4, in some implementations, the tension lines 114 may be proximally supported by, or anchored relative to, a support band 116 or reinforcement band positioned outside a patient incision when deployed. In this configuration, the support band 116 may provide a proximal reaction surface or anchor feature against which tension in the tension lines 114 may be maintained while the imaging module 20 and mounting structure 110 are positioned within the patient anatomy. In operation, the tension lines 114 may be tensioned and adjusted by drawing a free end 115 of each of the tension lines 114 through a locking collar 118. The locking collars 118 may correspond to spring-biased cinching devices configured to allow a tension line 114 to be pulled through a locking aperture 122 in response to an applied tensioning force 126 while resisting reverse motion to maintain the applied tension 126. The locking collar 118 may further comprise a release actuator 124 that, when actuated, permits the tensioning force 126 to be reduced or released (e.g., by disengaging a cam, pawl, collet, wedge, or friction interface). In this way, the adjustment angle θ of the one or more imagers 70 may be adjusted by varying the applied tension 126 by the opposing tension lines 114.

The locking collar 118 may be implemented in a variety of configurations including, but not limited to, a cord lock, a spring-loaded cam cleat, a one-way ratcheting clamp, a pawl-and-tooth cinch, a wedge-lock device, a collet-style clamp, a set-screw clamp, or combinations thereof. Additionally, the tension lines 114 may be implemented as cables, braided fibers, monofilament lines, sutures, woven tapes, polymer cords, metallic wires, or composite lines. For example, the tension lines 114 may comprise braided polyethylene fibers, braided polyester, nylon, polypropylene, aramid fibers, stainless steel cable, or shape-memory alloy wire (e.g., nitinol), including coated or jacketed variants to enhance abrasion resistance and biocompatibility.

As also illustrated in FIG. 4, a pivot plate 120 may be interposed between tissue of the patient (e.g., proximate the compression surface 50) and the mounting structure 110. In this configuration, the pivot plate 120 may provide an angled support surface 128 and/or a fulcrum-like support such that application of tension via the tension lines 114 allows opposing side portions 110a, 110b of the mounting structure 110 to translate relative to the pivot plate 120 in a teeter-totter or tipping configuration. For example, tension applied to the first tension line 114a may draw the first end portion 110a and cause the mounting structure 110 to tip in a first direction about the pivot plate 120, and tension applied to the second tension line 114b may draw the second end portion 110b and cause the mounting structure 110 to tip in a second direction about the pivot plate 120. In this way, the pitch of the imagers 70 and corresponding fields of view 60 may be adjusted in conjunction with the adjustment angle θ while the imaging module 20 is deployed. Similar to the operation of the pivot plate 120, it shall be understood that tipping and control of the adjustment angle θ may similarly be implemented by selectively tightening each of the tension lines 114 to compress the compressible tissue 14 and adjust the pitch of the adjustment angle θ in the corresponding direction of the one of the opposing side portions 110a, 110b.

Still referring generally to FIG. 4, the imaging module 20 may comprise a plurality of cameras 70 arranged such that corresponding fields of view 60a, 60b at least partially overlap. The overlapping arrangement of the fields of view 60 may enable a number of advantageous imaging modes, including selective activation of individual cameras 70, selective display of individual fields of view 60, and generation of composite image data formed by stitching or combining image data captured by two or more cameras 70. In some implementations, the composite image data may correspond to an extended field-of-view that is larger than the field-of-view provided by any single camera (e.g., 70a) alone.

The use of multiple cameras 70 with overlapping fields of view 60a, 60b, 60c may be particularly beneficial in the limited confines or constrained space formed by the passage 34, which may be created by displacing or retracting the compressible tissue 14 from the region of interest 30. In such confined environments, a single fixed field-of-view may not adequately capture all relevant anatomical features or surgical tools without repositioning the imaging apparatus 10. By selectively activating different cameras 70 or selectively displaying different fields of view 60, a surgeon or clinician may effectively inspect different portions of the region of interest 30 without physically moving the imaging module 20 or further disturbing the surrounding tissue.

In some implementations, a first camera 70a may be activated to provide a primary view aligned toward a central portion of the region of interest 30, while one or more additional cameras 70 may be selectively activated to provide auxiliary views oriented laterally, upward, downward, or at oblique angles relative to the mounting structure 110. In this configuration, the cameras 70 and the corresponding field-of-view 60 may be oriented to capture one or more neighboring or partially overlapping sections of the passage 34. In various implementations, image data and representative scenes from two or more cameras 70 may be stitched together to generate composite image data that may provide an expanded or panoramic view of the region of interest 30. In this way, the system 92 may support improved situational awareness or provide intuitive selections between or among fields-of-view 60 for the surgeon or clinician to effectively conduct a procedure within the confined space adjacent to the displaced compressible tissue 14.

In various examples, the selective activation and/or selective display of the scenes captured by the cameras 70 and corresponding fields of view 60a, 60b, 60c may further limit visual clutter or distraction by presenting the user with the most relevant image data for a particular procedural step. For example, during navigation of a surgical tool through or intersecting with the passage 34, a narrow or forward-looking field-of-view may be displayed, whereas during inspection or manipulation within the region of interest 30, a wider composite field-of-view may be generated by combining image data from multiple cameras 70. Such steps may be programmed or trained for a variety of procedures or based on the preference of the surgeon or clinician.

The selection of the one or more fields of view 60a, 60b, 60c may be user directed via a user interface and/or the imaging system 92 may be configured to detect motion or activity within one or more of the fields of view 60 by processing the image data captured by the cameras 70. In various implementations, activity may be detected by analyzing changes in pixel values, optical flow, frame-to-frame differences, edge movement, or other motion-based indicators within the image data. In some implementations, activity detection may further include detecting motion associated with one or more specified or predefined objects, such as surgical instruments, anatomical features, tissue boundaries, fluid flow, or other objects of interest within the passage 34 or the region of interest 30.

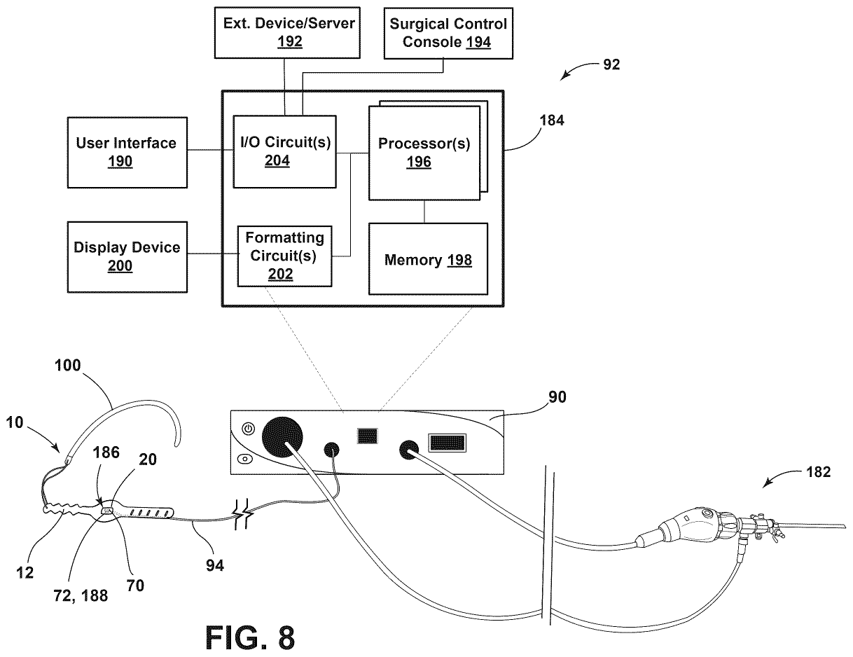

In response to detected activity, the controller 184 or control or processing circuits 186 (FIG. 8) may automatically select, prioritize, or activate one or more of the individual image data feeds corresponding to the fields of view 60 in which activity is detected. For example, when motion is detected in a first field-of-view 60a associated with manipulation of a surgical tool, the controller 184 may selectively display the image data from that field-of-view 60a or increase the prominence of that image data relative to other fields of view 60b, 60c. In other implementations, the controller 184 may selectively merge or stitch image data from multiple adjacent fields of view 60 to generate a panoramic or composite image depicting the spatial extent of the detected activity. In some implementations, the controller 184 may dynamically adjust proportions or format in which the image data is presented based on the detected activity. For example, the controller 184 may selectively crop, scale, zoom, pan, or otherwise modify the presentation of image data from one or more fields of view 60 to focus on the bounds or scope of the detected activity. Such presentation adjustments may be applied automatically based on the detected motion or in response to user input, and may further be constrained to user-defined regions corresponding to the region of interest 30 or other predefined zones within the passage 34.

In various implementations, activity-based processing may be combined with selective stitching or blending of overlapping image regions such that only the portions of the image data associated with detected activity are merged into a composite image. In this way, the imaging system may present a composite or panoramic view that emphasizes active regions while minimizing visual clutter from inactive portions of the fields of view 60. The controller 184 may further be configured to update the composite image dynamically as activity shifts between different fields of view 60 during the course of a procedure. Accordingly, motion- or activity-based processing of the image data may enable the imaging apparatus 10 to automatically adapt the selection, composition, and presentation of image data to the procedural context. This may further reduce cognitive load on the user, improve situational awareness within the constrained space formed by the passage 34, and enhance visualization of the region of interest 30 without requiring manual repositioning of the imaging apparatus 10.

As discussed herein, activity detection may be implemented using image processing techniques such as background subtraction, frame differencing, temporal filtering, optical flow estimation, edge or feature tracking, or combinations thereof to identify regions of motion within the image data. In some implementations, trained models may be utilized to detect motion associated with one or more specified or predefined objects, including surgical instruments, tool tips, tissue surfaces, anatomical landmarks, fluid flow, or other objects of interest, for example using object detection, segmentation, or keypoint-based models trained on labeled image data. Outputs of such processing may be used by the controller 184 to automatically select one or more image feeds corresponding to fields of view 60 in which activity is detected, selectively merge or stitch image data from adjacent fields of view 60 to generate a panoramic or composite image emphasizing active regions, and selectively crop, scale, zoom, pan, or otherwise present the image data to focus on the bounds or scope of detect

Referring now to FIG. 5, a projected view of the elongated band 12 of the imaging apparatus 10 is illustrated in a simulated deployed state. As illustrated, the elongated band 12 may be curved in a shape representative of the band 12 enclosed about the compressible tissue 14 when deployed in a procedure. In this configuration, the curvature of the band 12 corresponds generally to the path of the passage 34 formed around the compressible tissue 14 and adjacent the region of interest 30. In reference to this exemplary deployed condition, various aspects of the imaging apparatus 10 are demonstrated and described that may be implemented individually or in various combinations to support the operation of the system 92.

As demonstrated in FIG. 5, examples of the imaging apparatus 10 implemented for the imaging module 20 are shown. In the illustrated examples, the imaging apparatus 10 may be modified to include multiple cameras 70 (e.g., cameras 70a, 70b, 70c) with corresponding fields of view 60. The cameras 70 may be arranged in a linear array along the length of the elongated band 12 or in other configurations as later described. Each camera 70 may be configured to capture image data within a respective field-of-view 60, which may partially overlap with one or more adjacent fields of view 60.

As further demonstrated in DETAILS C and D of FIG. 5, examples of the cameras 70 are shown connected to a mounting structure 110, which may correspond to one or more printed circuit boards (PCBs) or similar support substrates. The mounting structure 110 may be implemented as a single continuous structure or as multiple coupled portions that are connected in a hinged, flexible, or bendable configuration. Accordingly, the cameras 70 may be configured to vary in direction relative to one another. As shown, the mounting structure 110 may include a hinge, bend, or flex section 136 along which coupled portions 138a and 138b of the mounting structure 110 are connected. In this configuration, focal axes A_{f} of the cameras 70 may be selectively angled or adjusted along an interposed alignment angle φ responsive to the bending, flexing, or pivoting at the flex section 136. For example, adjacent cameras 70 may be oriented with alignment angles φ of approximately 5°, 10°, 15°, 20°, 30°, or greater relative to each another. As shown, the focal axes A_{f} may include a first focal axis A_{f1}, a second focal axis A_{f2}, and/or a third focal axis A_{f3}, which may correspond the focal axes A_{f} of the first camera 70a, second camera 70b, and/or third camera 70c, respectively.

In some implementations, the coupled portions 138a, 138b of the mounting structure 110 may be adjusted to one or more angled configurations and fixed by locking or biasing the flex section 136. In other implementations, the flex section 136 may be left free to bend or flex such that the coupled portions 138a, 138b and corresponding cameras 70 may generally conform to the curvature or shape of the elongated band 12 along the compression surface 50. In such cases, the focal axes A_{f} and corresponding fields of view 60 of the cameras 70 may be oriented approximately normal to the curvature of the compression surface 50 and may generally direct the fields of view 60 away from the compression surface 50. The flex section 136 may be implemented using a variety of hinge or bendable structures, which may include one or more elastomeric hinge regions, pinned hinges, or compliant joints. In some implementations, the flex section 136 may comprise a flexible or bendable material such as a polymeric substrate, which may correspond to an elastomer or laminated composite, or a shape-memory material. For example, the flex section 136 may be configured to bend under applied forces but maintains a bias (e.g., via a spring force) toward a predetermined orientation of the alignment angle φ (e.g., 5° or 10°) in a predefined shape or angle, which may achieved by utilizing a shape-memory alloy or shape-memory polymer configured to return to a the predetermined orientation of the alignment angle φ.

As may be clear from the corresponding illustrations in DETAIL C, the number of the coupled sections 138 may vary and may include a third coupled support section 138c, and each of the coupled sections may or may not include a corresponding camera 70. Accordingly, the coupled sections 138 may be operably coupled by corresponding flex sections and may be bent, flex, or adjust along corresponding alignment angles φ, as shown including a first alignment angle φ₁ and a second alignment angle φ₂. Additionally, any of the first camera 70a, the second camera 70b, and/or the third camera 70c may be omitted from one or more of the coupled sections 138, which may provide for the coupled sections to periodically or selectively form intermediate linking sections between or among the cameras 70. As previously described, the number of cameras 70 may vary widely in number, spacing, and position to suit various applications. Accordingly, the disclosure may provide for a variety of configurations that may be implemented alone or in any combination to support the operations disclosed.

In conjunction with the alignment angle φ or angles, the fields-of-view 60 of the cameras 70 may oriented in a predetermined configuration. For example, adjacent instances of the cameras 70 may be aligned (e.g., fixedly or flexibly) at a relative alignment angle φ of approximately 1°-10° to provide incremental coverage along the length of the passage 34, or at approximately 1° −20° to provide a wider composite field-of-view. In such configurations, the relative spacing between the cameras 70 and the corresponding alignment angles φ may be selected to ensure that the respective fields of view 60 substantially cover or effectively capture image data along a corresponding length of the passage 34. By appropriately selecting the alignment angles φ and camera spacing, intervening segments of the passage 34 that are not captured within the fields of view 60 may be reduced or eliminated. More generally, the cameras 70 may be arranged with a variety of angular relationships relative to one another. For example, focal axes A_{f} of adjacent cameras 70 may diverge at alignment angles φ of approximately 10°-30° to increase lateral coverage, converge at alignment angles φ of approximately 2°-8° to support stereoscopic or multiscopic depth imaging, or be arranged at mixed angles to balance depth perception and longitudinal coverage. Such angular relationships may be selected to support visualization along the path of the passage 34 and, in particular, within the region of interest 30.

Additionally, the cameras 70 may be arranged in a linear array, a two-dimensional matrix, or other array configurations. In matrix arrangements, neighboring rows or columns of cameras 70 may be angled relative to one another in a manner similar to that described above with reference to the alignment angles φ. Accordingly, the imaging apparatus 10 may include two, three, four, or more cameras 70 arranged in an array, matrix, and/or variably spaced configuration along the length of the elongated band 12 depending on the intended application. In some implementations, the cameras 70 may further include optical assemblies selected to maintain coverage of the image data captured along the passage 34 and/or within the region of interest 30. For example, the cameras 70 may include lenses having viewing angles of approximately 60°, 90°, 120°, or greater. In some implementations, wide-angle or fisheye lenses (e.g., 140°-180° diagonal field-of-view) may be utilized to increase angular coverage, compensate for curvature of the elongated band 12, and enhance overlap between adjacent fields of view 60.

As further demonstrated in the example of FIG. 5, the cameras 70 may be electronically and/or structurally coupled to one another via flexible connections 142 extending along or within the mounting structure 110. The flexible connections 142 may include flexible printed circuit traces, ribbon cables, braided conductors, stretchable interconnects, or other compliant electrical interfaces that may maintain electrical connectivity between or among the cameras 70 while permitting relative movement or bending of the mounting structure 110. In this way, the imaging apparatus 10 may provide both structural adaptability and electrical communication among the cameras 70 when deployed for a variety of applications.

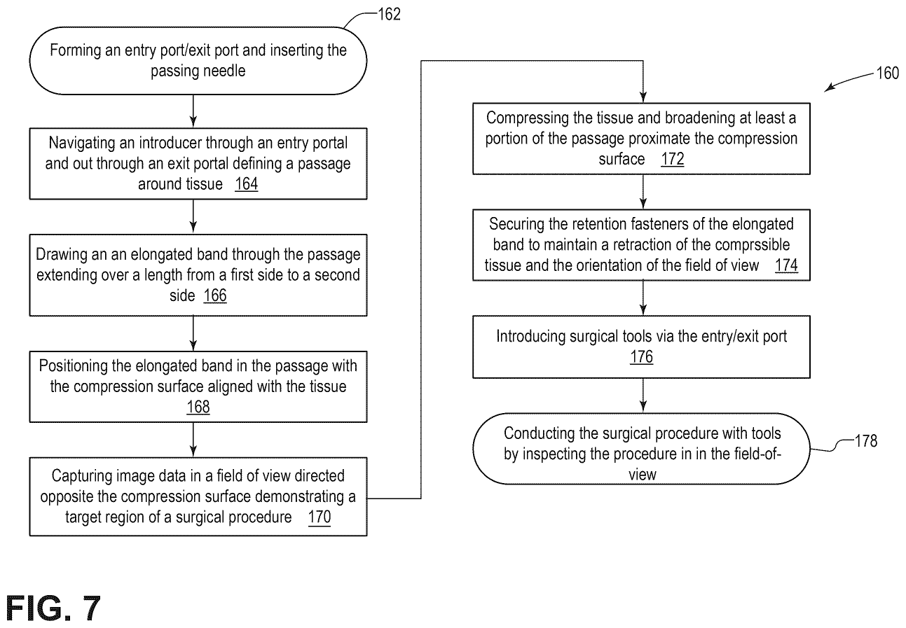

Referring now to FIGS. 6A-6D, exemplary procedural steps for deploying the imaging apparatus 10 are shown in reference to a knee joint 16. Additionally, the corresponding procedural steps for a method 160 of executing the procedure demonstrated in FIGS. 6A-6D may similarly be represented in the flow chart of FIG. 7. As demonstrated in FIG. 6A, the method 160 may begin by inserting the distal end portion 102 of the passing needle 100 into the entry port 42 (162). As shown, the entry port 42 and exit port 44 may be aligned based on a surgical plan, such that the path of the passing needle 100 forming the passage 34 may extend through the anatomy of interest, in this case the knee joint 16, on an opposing side to form a loop around the compressible tissue 14 (164). As shown in FIG. 6B, the path forming the passage 34 may extend from the entry port 42 to the exit port 44.

As demonstrated in FIG. 6C, the method 160 may continue by drawing the elongated band 12 through the passage 34 between the entry port 42 and the exit port 44 (166). With the elongated band 12 positioned within the passage 34, the method 160 can continue by positioning the compression surface 50 aligned with the compressible tissue 14 (168). At this stage in the method 160, the imaging module 20 may be activated and/or the imaging data captured in the field-of-view 60 may be reviewed to assist in positioning the elongated band 12 along the length L within the passage 34 (170). By reviewing the image data within the field-of-view 60, the field-of-view 60 may be aligned with the region of interest 30 associated with the procedure.

Still referring to FIGS. 6 and 7, with the field-of-view 60 aligned with the region of interest 30, the method 160 can continue by securing the elongated band 12 to the patient and compressing the compressible tissue 14 extending along the compression surface 50 and through the passage 34 (172). With the compressible tissue 14 retained by the elongated band 12 and the field-of-view 60 aligned with the region of interest 30, the method 160 may continue by securing the retention fasteners of the elongated band to maintain the retraction of the compressible tissue 14 and the positioning of the field-of-view 60 (174). The elongated band 12 of the imaging apparatus 10 may be shown enclosed about the compressible tissue 14 in FIG. 6D.

With the imaging apparatus 10 secured to the patient, the surgical procedure may continue by introducing one or more surgical tools through the entry port 42 and/or the exit port 44 (176). In this configuration, a surgeon or medical professional may conduct various steps of the surgical procedure while viewing the image data in the field-of-view 60 (178). Accordingly, the imaging apparatus 10 may provide for the beneficial retraction of the compressible tissue 14 to expand an operating volume proximate the region of interest 30 as well as capture the image data depicting the field-of-view 60. The imaging apparatus 10 may further be implemented in combination with one or more additional scopes (e.g., arthroscopes, endoscopes, laparoscopes) to provide a consistent view of the region of interest 30 without requiring the surgeon or a surgical team to actively maneuver a scope through the entry and/or exit ports 42, 44 throughout the entire procedure.

Referring now to FIG. 8, a block diagram of an imaging system 92 associated with the imaging apparatus 10 is shown. In various implementations, the system 92 may correspond to a video control console in communication with a surgical scope 182 and/or the imaging apparatus 10. The system 92 may be further in communication with various surgical tools via the controller 184. The surgical scope 182 may correspond to various devices including an endoscope, laparoscope, arthroscope, etc. The imaging apparatus 10 may correspond to a chip-on-wire imaging device comprising control circuitry 186 incorporated in the imaging module 20 of the imaging apparatus 10. As demonstrated, the surgical scope 182 and the imaging apparatus 10 may be in communication with the controller 184 via a communication interface. Though shown connected via a conductive connection, the communication interface may correspond to a wireless communication interface operating via one or more wireless communication protocols (e.g., Wi-Fi, 802.11 b/g/n, etc.).

In various implementations, the imaging apparatus 10 may include the light source 72 as one or more emitters 188, which may correspond to various light emitters configured to generate light in the visible range, the near infrared range, or various wavelengths. In various implementations, the emitters 188 may include light emitting diodes (LEDs), laser diodes, or other lighting technologies. The image sensor(s) or imager 70 may correspond to various sensors and configurations comprising, for example, complementary metal-oxide semiconductor (CMOS) sensors, or similar sensor technologies. In some implementations, the imaging apparatus 10 may include an inertial sensor (e.g., accelerometer, inertial measurement unit, gyroscope, magnetometer etc.), which may supply orientation signals (e.g., gravitational direction, field directions, etc.) to the controller 184. In this configuration, the controller 184 may track and offset the orientation of the field-of-view (FOV) 60 of the imaging apparatus 10 relative to gravity or another prevailing force in real time or with minimal delay associated with a framerate (e.g., 30 frames per second [FPS], 60 FPS, 90 FPS) of the image feed captured by the imager 70. In this configuration, a controller 184 may adjust an orientation of the image data, such that the anatomy or features in the field-of-view 60 are consistently presented relative to gravity or a predefined offset relative to gravity. Accordingly, the controller 184 of the imaging system may process image data responsive to the orientation of the imager 70 to present the features of the operating environment consistently with respect to gravity or other prevailing/detectable forces or signals. For example, while gravity is mentioned specifically, other prevailing signals (e.g., artificial or natural magnetic fields), radio frequency signals, or similar signals may be detected to consistently orient the image data relative to gravity or another direction of interest.

In various implementations, the imaging apparatus 10 may comprise the control circuitry 186 configured to control the operation of imager 70 and the emitter(s) 208 as well as process and/or communicate the image data to the controller 184. Additionally, the control circuitry 186 may be in communication with a user interface 190, which may include one or more input devices, indicators, displays, etc. The user interface 190 may provide for the control of the imaging apparatus 10 including the activation of one or more control routines. The user interface 190 may provide for the selection, adjustment, or toggling of one or more of the image feeds associated with the operation of the imaging apparatus 10 and/or the scope 182. The control circuitry 186 may be implemented by various forms of controllers, microcontrollers, application-specific integrated controllers (ASICs), and/or various control circuits or combinations.

The controller 184 or system controller may comprise a processor 196 and a memory 198. The processor 196 may include one or more digital processing devices including, for example, a central processing unit (CPU) with one or more processing cores, a graphics processing unit (GPU), digital signal processors (DSPs), field programmable gate arrays (FPGAs), application-specific integrated circuits (ASICs) and the like. In some configurations, multiple processing devices are combined into a System-on-a-Chip (SoC) configuration while in other configurations the processing devices may correspond to discrete components. In operation, the processor 196 executes program instructions stored in the memory 198 to perform various operations related to the operation of the imaging system 92 as well as one or more surgical control consoles in communication with the controller 184.

The memory 198 may comprise one or more data storage devices including, for example, magnetic or solid-state drives and random access memory (RAM) devices that store data. The memory 198 may include one or more stored program instructions, object detection templates, image processing algorithms, etc. In various implementations, the controller 184 may correspond to a display or video controller configured to output formatted image data to one or more display devices 200. In such applications, the controller 184 may include one or more formatting circuits 202, which may process the image data received from the imaging apparatus 10 and/or the surgical scope 182, communicating with the processor 196, and processing the image data for presentation on the one or more display devices 200. The formatting circuits 202 may include one or more signal processing circuits, analog-to-digital converters, digital-to-analog converters, etc. The user interface 190 of the controller 184 may be in the form of an integrated interface (e.g., a touchscreen, input buttons, an electronic display, etc.) or may be implemented by one or more connected input devices (e.g., a tablet) or peripheral devices (e.g., keyboard, mouse, foot pedal, etc.).

As shown, the controller 184 may also be in communication with an external device or server 192, which may correspond to a network, local or cloud-based server, device hub, central controller, or various devices that may be in communication with the controller 184 and, more generally, the imaging system 92 via one or more wired (e.g., serial, Universal Serial Bus (USB), Universal Asynchronous Receiver/Transmitter (UART), etc.) and/or wireless communication interfaces (e.g., a ZigBee, an Ultra-Wide Band (UWB), Radio Frequency Identification (RFID), infrared, Bluetooth®, Bluetooth® Low Energy (BLE), Near Field Communication (NFC), etc.) or similar communication standards or methods. For example, the controller 184 may receive updates to the various modules and routines as well as communicate sample image data from the imaging apparatus 10 to a remote server for improved operation, diagnostics, and updates to the imaging system 92. The user interface 190, the external server 192, and/or a surgical control console 194 may be in communication with the controller 184 via one or more I/O circuits 204. The I/O circuits 204 may support various communication protocols including, but not limited to, Ethernet/IP, TCP/IP, Universal Serial Bus, Profibus, Profinet, Modbus, serial communications, etc.

According to some aspects of the disclosure, a surgical imaging apparatus includes an elongated band extending over a length from a first side to a second side with an intermediate section defining a compression surface formed therebetween. An imaging module is operably coupled to the intermediate section and configured to capture image data in a field-of-view directed from a viewing surface of the intermediate section.

According to various aspects, the disclosure may implement one or more of the following features or configurations in various combinations:
a passing needle is operably coupled to the first side of the elongated band;
the passing needle is operably coupled via a flexible coupling material;
the passing needle comprises a blunt elongated body having a tapered tip;
the elongated body defines a passing diameter that is greater than 1 mm;
the elongated band is a compression band configured to close about and retract soft or compressible tissue;
the elongated band comprises opposing retention fasteners configured to interconnect on the first side and the second side;
the opposing retention fasteners comprise a plurality of receiving slots extending along a first portion of the length and a plurality engaging ribs that engages the receiving slots;
the engaging ribs form wings that extend on opposing sides of the length of the elongated band;
the closure of the elongated band directs the field-of-view to an operation space within a patient anatomy from a position occupied by the soft or compressible tissue in a natural or uncompressed state;
the viewing surface is approximately opposite the compression surface;
the field-of-view is directed away from the compression surface;
at least one light source configured to output a light emission in the field-of-view;
a communication interface operably coupled to the second side and interconnecting the imager; and/or
the communication interface extends along the length of the second side and is in connection with a cable connector configured to communicate the image data captured in the field-of-view to a camera controller.

According to another aspect of the disclosure, a method of deploying a camera in connection with the tissue of a patient includes the steps of: inserting an elongated, curved structure through an entry portal and an exit portal defining a passage around the tissue; drawing an elongated band through the passage extending over a length from a first side to a second side with an intermediate section defining a compression surface formed therebetween; passing the elongated band through the passage positioning the compression surface with the tissue; compressing the tissue and broadening at least a portion of the passage proximate the compression surface; and capturing image data in a field-of-view directed into the passage (e.g., approximately opposite the compression surface). The image data demonstrates a target region of a surgical procedure.

According to various aspects, the disclosure may implement one or more of the following features or configurations in various combinations:
the positioning of the compression surface also adjusts the position of the field-of-view;
conducting the surgical procedure with one or more elongated surgical tools, wherein a navigation of the one or more elongated surgical tools is captured in the field-of-view;
providing an imaging apparatus configured to capture the image data in the field-of-view in connection with the elongated band proximate the compression surface;
the compression of the tissue comprises connecting a first mating feature of the first side to a second mating feature of the second side, thereby maintaining the compression of the tissue and the position of the field-of-view;
the tissue comprises soft or compressible tissue;
the elongated, curved structure comprise a blunt-tipped needle; and/or
a tip of the blunt-tipped needle comprises a bullet shape.

As used herein, words of approximation such as, without limitation, “approximately,” “substantially,” or “about” refer to a condition that when so modified is understood to not necessarily be absolute or perfect but would be considered close enough to those of ordinary skill in the art to warrant designating the condition as being present. Further, the lack of such modifying terms does not otherwise require strict interpretation of the corresponding value or property. Instead, the extent to which the associated interpretation varies will depend on how great a change can be instituted and still have one of ordinary skill in the art recognize the modified feature as having the required characteristics or capabilities of the unmodified feature. Such determinations may vary considerably depending on the technological field based on the corresponding equivalency associated with the described operation or property. In general, but subject to the preceding discussion, a numerical value herein that is modified by a word of approximation such as “approximately” may vary from the stated value by ±0.5%, ±1%, ±2%, ±3%, ±4%, ±5%, ±10%, ±12%, or ±15%.

Any element in a claim that does not explicitly state “means” for performing a specified function or “step” for performing a specified function, should not be interpreted as a “means” or “step” clause as specified in 35 U.S.C. § 112.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the embodiments shown in the drawings and described above are merely for illustrative purposes and not intended to limit the scope of the device, which is defined by the following claims as interpreted according to the principles of patent law, including the Doctrine of Equivalents.

1. A surgical imaging apparatus comprising:
an elongated band extending over a length from a first side to a second side with an intermediate section defining a compression surface formed therebetween; and
an imaging module operably coupled to the intermediate section and configured to capture image data in a field-of-view directed from a viewing surface of the intermediate section.

2. The surgical imaging apparatus according to claim 1, wherein a passing needle is operably coupled to the first side of the elongated band.

3. The surgical imaging apparatus according to claim 2, wherein the passing needle is operably coupled via a flexible coupling material.

4. The surgical imaging apparatus according to claim 3, wherein the passing needle comprises a blunt elongated body having a tapered tip.

5. The surgical imaging apparatus according claim 1, wherein the elongated body defines a passing diameter that is greater than 1 mm.

6. The surgical imaging apparatus according to claim 1, wherein the elongated band is a compression band configured to close about and retract soft or compressible tissue.

7. The surgical imaging apparatus according to claim 1, wherein the elongated band comprises opposing retention fasteners configured to interconnect on the first side and the second side.

8. The surgical imaging apparatus according to claim 7, wherein the opposing retention fasteners comprise a plurality of receiving slots extending along a first portion of the length and a plurality engaging ribs that engages the receiving slots.

9. The surgical imaging apparatus according to claim 8, wherein the engaging ribs form wings that extend on opposing sides of the length of the elongated band.

10. The surgical imaging apparatus according to claim 1, wherein the closure of the elongated band directs the field-of-view to an operation space within a patient anatomy from a position occupied by the soft or compressible tissue in a natural or uncompressed state.

11. The surgical imaging apparatus according to claim 1, wherein the viewing surface is approximately opposite the compression surface.

12. The surgical imaging apparatus according to claim 1, wherein the field-of-view is directed away from the compression surface.

13. The surgical imaging apparatus according to claim 1, further comprising at least one light source configured to output a light emission in the field-of-view.

14. The surgical imaging apparatus according to claim 1, further comprising:
a communication interface operably coupled to the second side and interconnecting the imager.

15. The surgical imaging apparatus according to claim 14, wherein the communication interface extends along the length of the second side and is in connection with a cable connector configured to communicate the image data captured in the field-of-view to a camera controller.

16. A method of deploying a camera in connection with the tissue of a patient, the method comprising:
inserting an elongated, curved structure through an entry portal and an exit portal defining a passage around the tissue;
drawing an elongated band through the passage extending over a length from a first side to a second side with an intermediate section defining a compression surface formed therebetween;
passing the elongated band through the passage positioning the compression surface with the tissue;
compressing the tissue and broadening at least a portion of the passage proximate the compression surface; and
capturing image data in a field-of-view directed into the passage (e.g., approximately opposite the compression surface), wherein the image data demonstrates a target region of a surgical procedure.

17. The method according to claim 16, wherein the positioning of the compression surface also adjusts the position of the field-of-view.

18. The method according to claim 16, further comprising:
conducting the surgical procedure with one or more elongated surgical tools, wherein a navigation of the one or more elongated surgical tools is captured in the field-of-view.

19. The method according to claim 16, further comprising:
providing an imaging apparatus configured to capture the image data in the field-of-view in connection with the elongated band proximate the compression surface.

20. A surgical imaging apparatus comprising:
an elongated band extending over a length from a first side to a second side and configured to be positioned around compressible tissue to form a passage adjacent a region of interest;
an imaging module operably coupled to the elongated band;
at least one camera of the imaging module positioned along the length of the elongated band and configured to capture image data in a corresponding field-of-view directed generally away from a compression surface of the elongated band;
at least one tensioning line coupled to a support plate of the at least one camera, wherein selective application of tension to the tensioning line causes the support plate to pivot and adjust a pitch of the field-of-view of the at least one camera; and
a controller configured to receive image data from the at least one camera and selectively output image data from one or more of the fields of view or composite image data generated from a combination of the image data from two or more of the cameras.
